(19) United States
(12) Patent Application Publication
(10) Pub. No.: US 2022/0265520 A1
(43) Pub. Date: Aug. 25, 2022

(54) AQUEOUS LIQUID COSMETIC AND PEN-SHAPED COSMETIC PRODUCT

(71) Applicant: TOKIWA CORPORATION, Nakatsugawa-shi (JP)

(72) Inventor: Kazunori OGINO, Kawaguchi-shi (JP)

(21) Appl. No.: 17/676,224

(22) Filed: Feb. 21, 2022

(30) Foreign Application Priority Data
Feb. 24, 2021 (JP) 2021-027523
Dec. 23, 2021 (JP) 2021-209264

(51) Int. Cl. A61K 8/02; A61K 8/20; A61K 8/24; A61K 8/29; A61K 8/88; A61K 8/81; A61Q 1/10

(52) U.S. Cl. CPC A61K 8/0204; A61K 8/20; A61K 8/24; A61K 8/29; A61K 8/88; A61K 8/8152; A61Q 1/10; A61K 2800/43; A61K 2800/5424; A61K 2800/87

(57) ABSTRACT

An aqueous liquid cosmetic contains a pigment, a dispersant, and an inorganic salt, and the cosmetic has a viscosity of 1 to 50 mPas at 25° C. The pigment contains at least one selected from the group consisting of Prussian blue and lakes of natural colorants, and the inorganic salt contains at least one selected from the group consisting of a chloride, a sulfate, a nitrate, a carbonate, and a phosphate.

### CROSS-REFERENCE TO RELATED APPLICATIONS

This application claims the benefit of priority from Japanese Patent Application No. 2021-027523, filed on Feb. 24, 2021, and Japanese Patent Application No. 2021-209264, filed on Dec. 23, 2021, the entire contents of which are incorporated herein by reference.

### TECHNICAL FIELD

The present invention relates to an aqueous liquid cosmetic and a pen-shaped cosmetic product.

### BACKGROUND

As a product form of aqueous liquid cosmetics, automatic pen-shaped applicators have been utilized from the viewpoint of ease of use. In these applicators, a cosmetic is caused to follow from an accommodation unit filled with the cosmetic to a pen tip by a capillary phenomenon. The cosmetic used in this case often has low viscosity from the viewpoint of flowability, and pigments are likely to sediment over time. The sedimentation of pigments leads to the problems of clogging of the accommodation unit and flow channel of the cosmetic, and of blurring of drawn lines. For the purpose of suppressing such problems, enhancement of the dispersibility and dispensability of an aqueous liquid cosmetic has been hitherto achieved by blending an anionic dispersant such as sodium polyaspartate to the cosmetic (see, for example, Japanese Unexamined Patent Publication No. 2003-231614 and Japanese Unexamined Patent Publication No. 2015-164912).

### SUMMARY

Regarding liquid eyeliners, eyebrow cosmetics, and the like, development of various colors such as blue, green, and milky colors in addition to black and brown colors, has been seen increasingly in recent years. Apart from conventional black pigments such as black iron oxide and carbon black, there may be used for occasional admixing, inorganic pigments (e.g., red iron oxide, yellow iron oxide, titanium oxide, and Prussian blue) and synthetic organic pigments (e.g., coal-tar color and lakes of natural colorants such as carmine).

However, the inventors found that in aqueous liquid cosmetics including Prussian blue or a lake of a natural colorant, color separation occurs at the pen tip of an applicator. Such color separation at the application part leads to shading difference (or hue difference) of drawn lines, which is not desirable for cosmetic products. It was also found that the color separation cannot be sufficiently suppressed by blending of an anionic dispersant to the product.

Disclosed herein is an aqueous liquid cosmetic that has sufficient dispensability even when used for an automatic pen-shaped applicator and that does not easily undergo color separation at the application part despite the inclusion Prussian blue or a lake of a natural colorant. Disclosed also is a pen-shaped cosmetic product containing the aqueous liquid cosmetic.

In some examples, the aqueous liquid cosmetic comprises (A) a pigment, (B) a dispersant, and (C1) an inorganic salt, the cosmetic having a viscosity of 1 to 50 mPa·s at 25° C., wherein the component (A) comprises (A1) at least one selected from the group consisting of Prussian blue and lakes of natural colorants, and the component (C1) comprises at least one inorganic salt selected from the group consisting of a chloride, a sulfate, a nitrate, a carbonate, and a phosphate.

According to the aqueous liquid cosmetic having the composition described above, sufficient discharge is enabled even when the aqueous liquid cosmetic is used for an automatic pen shaped applicator, and at the same time, color separation at the application part can be suppressed.

From the viewpoints of suppressing color separation at the application part and of dispensability, the aqueous liquid cosmetic may comprise a chloride as the component (C1), and a content of the chloride may be 0.05% to 1% by mass based on a total amount of the cosmetic.

From the viewpoints of suppressing color separation at the application part and of dispensability, the aqueous liquid cosmetic may comprise a polyphosphate as the component (C1), and a content of the polyphosphate may be 0.005% to 0.1% by mass based on a total amount of the cosmetic.

Furthermore, the component (B) may comprise (B1) an anionic dispersant.

From the viewpoint of achieving both the dispensability and the suppression of color separation at the application part at high levels, the aqueous liquid cosmetic may comprise (B1-L) an anionic polymer having an average molecular weight (Mw) of 1,000 to 30,000 as the component (B1), and a mass ratio of the component (C1) to the component (B1-L), [(C1)/(B1-L)], may be 0.3/5 to 3/1.

From the viewpoint of achieving all the dispensability and the suppression of color separation at the application part and the shading difference in the drawn lines, the aqueous liquid cosmetic may comprise as the component (B1), (B1-L) an anionic polymer having an average molecular weight (Mw) of 1,000 to 30,000 and (B1-H) an anionic polymer having an average molecular weight (Mw) of 50,000 to 1,000,000, as the component (B1).

The component (A) may further comprise (A2) at least one metal oxide selected from the group consisting of titanium oxide, red iron oxide, yellow iron oxide, black iron oxide, and titanium black.

Disclosed further is a pen-shaped cosmetic product comprising an automatic pen shaped applicator; and the above-described aqueous liquid cosmetic filled in the applicator.

### BRIEF DESCRIPTION OF THE DRAWINGS

FIG. 1 is a schematic cross-sectional view for describing a pen-shaped cosmetic product.

### DETAILED DESCRIPTION

An example aqueous liquid cosmetic comprises: (A) a pigment (hereinafter, may be referred to as component (A)); (B) a dispersant (hereinafter, may be referred to as component (B)); and (C1) at least one inorganic salt selected from the group consisting of a chloride, a sulfate, a nitrate, a carbonate, and a phosphate (hereinafter, may be referred to as component (C1)).

In one or more embodiments, the term “aqueous” may be understood to mean that at least water is contained in the liquid cosmetic. The aqueous cosmetic may further comprise a lower alcohol having 1 to 5 carbon atoms, such as ethanol, in addition to water.

In one or more embodiments, a content of water in the aqueous liquid cosmetic may be 30% to 85% by mass, may be 40% to 80% by mass, or may be 50% to 70% by mass, based on a total amount of the cosmetic.

In one or more embodiments, the term “liquid” may be understood to refer to one having fluidity at 25 ° C. The viscosity at 25° C. of the aqueous liquid cosmetic can be set to 1 to 50 mPa·s at 25 ° C.

From the viewpoint of obtaining satisfactory dispensability in the automatic pen-shaped applicator, the aqueous liquid cosmetic may have a viscosity of 40 mPa·s or less, 35 mPa·s or less, 30 mPa·s or less, or 25 mPa·s or less at 25° C. Also, from the viewpoint of usability, the aqueous liquid cosmetic may have a viscosity of 4 mPa s or more, or 6 mPa·s or more at 25° C.

The above-described viscosity means a value measured for a sample at 25° C. under the following conditions using a Brookfield type viscometer (BM type).

1 to 50 mPa·s: BL adaptor, speed of rotation 12 rpm

(A) Pigment

Any pigment that is usually used for cosmetic products can be used. Examples thereof include inorganic pigments such as red iron oxide, yellow iron oxide, black iron oxide, cobalt oxide, chromium oxide, Ultramarine blue, Prussian blue, titanium oxide, microparticulate titanium oxide, zinc oxide, titanium black (titanium and titanium oxide sintered product), carbon black, barium sulfate, and pearl pigments (titanated mica, iron oxide-coated titanated mica, microparticulate titanium oxide-coated titanated mica, barium sulfate-coated titanated mica, fish scale guanine, bismuth oxychloride, aluminum flakes, and the like); organic pigments of synthetic colorants such as zirconium, barium, or aluminum lakes, such as Red No. 201, Red No. 202, Red No. 205, Red No. 226, Red No. 228, Orange No. 203, Orange No. 204, Blue No. 404, Yellow No. 401, Red No. 3, Red No. 104, Red No. 106, Orange No. 205, Yellow No. 4, Yellow No. 5, Green No. 3, and Blue No. 1; and products obtained by converting natural colorants such as carminic acid, laccaic acid, carthamin, brazilin, and crocin to the lakes of the foregoing (hereinafter, also referred to as “lakes of natural colorants”). These pigments may be subjected to a surface treatment for the purpose of enhancing usability, dispersibility, and the like. Examples of the surface treatment involve a metal soap, a silicone compound, a fluorine compound, a surfactant, and an amino acid compound.

Regarding the component (A), one kind thereof can be used singly, or two or more kinds thereof can be used in combination.

The aqueous liquid cosmetic may comprise, as the component (A), (A1) at least one selected from the group consisting of Prussian blue and lakes of natural colorants (hereinafter, may be referred to as component (A1)), from the viewpoint of developing various colors.

From the viewpoints of dispensability and color developability of drawn lines, a content of the component (A1) may be 1% to 99% by mass, may be 5% to 95% by mass, may be 10% to 90% by mass, or may be 20% to 85% by mass, based on a total amount of the component (A).

In one or more embodiments, the aqueous liquid cosmetic may further comprise as the component (A), at least one metal oxide selected from the group consisting of titanium oxide, red iron oxide, yellow iron oxide, black iron oxide, and titanium black (titanium-titanium oxide sintered product) (hereinafter, may be referred to as component (A2)), from the viewpoint of developing various colors.

In one or more embodiments, the aqueous liquid cosmetic can suppress color separation at the application part even when it contains the component (A1) and the component (A2). When the aqueous liquid cosmetic contains a white pigment as in the case where titanium oxide is blended in as the component (A2), color difference due to color separation at the application part tends to become more apparent; however, according to the aqueous liquid cosmetic, color separation at the application part can be sufficiently suppressed.

In one or more embodiments, a content of the component (A) in the aqueous liquid cosmetic may be 0.1% to 30% by mass, may be 1% to 25% by mass, or may be 2% to 20% by mass, based on a total amount of the cosmetic.

(B) Dispersant

Regarding the component (B), any dispersant that is usually used for cosmetic products may be used, and it can employ an anionic dispersant (hereinafter, may be referred to as component (B1)), a nonionic dispersant, or a water-soluble polymer other than these dispersants. Furthermore, a water-soluble compound may also be used as the component (B).

Examples of the component (B1) include anionic polymers such as polycarboxylates, including a polyaspartate, a polyacrylate, and a polymethacrylate. Furthermore, a water-soluble compound may also be used as the component (B1). Regarding the component (B1), one kind thereof can be used singly, or two or more kinds thereof can be used in combination.

A polyaspartate is a polymer including aspartic acid as a constituent unit. A polycarboxylate is a homopolymer or copolymer including a constituent unit derived from an ethylenically unsaturated carboxylic acid such as acrylic acid, methacrylic acid, maleic acid, or itaconic acid. A polyacrylate is a homopolymer including acrylic acid as a constituent unit, and a polymethacrylate is a homopolymer including methacrylic acid as a constituent unit.

Regarding a salt of the polymer, there may be used an alkali metal salt, such as a sodium salt or a potassium salt, an organic amine salt, such as a diethanolamine salt or a triethanolamine salt, and a basic amino acid salt. The salt of the polymer may be a sodium salt or a potassium salt.

The average molecular weight (Mw) of the anionic polymer may be 1,000 to 30,000, may be 1,000 to 15,000, or may be 2,000 to 10,000, from the viewpoint of achieving all the dispensability, the suppression of color separation at the application part, and the shading difference in the drawn lines. The average molecular weight (Mw) of the water-soluble polymer can be measured using gel permeation chromatography (GPC) by employing polyethylene glycols having known molecular weights as reference materials.

Regarding the component (B1), a commercially available product can be used. Examples of the polyaspartate include AQUADEW SPA-30 (sold under this name and manufactured by Ajinomoto Co., Inc., average molecular weight (Mw) 4,000) and Baypure DS100 (sold under this name and manufactured by Lanxess AG, average molecular weight (Mw) 2500). Examples of the polyacrylate include ARON T-50 (sold under this name and manufactured by Toagosei Co., Ltd., average molecular weight (Mw) 6,000) and ARON A-30SL (sold under this name and manufactured by Toagosei Co., Ltd., average molecular weight (Mw) 6,000). Examples of the polycarboxylate include ARON A-6330 (sold under this name and manufactured by Toagosei Co., Ltd., average molecular weight (Mw) 10,000) and ARON A-6001 (sold under this name and manufactured by Toagosei Co., Ltd., average molecular weight (Mw) 8,000).

The content of the component (B1) may be 0.05% to 5% by mass, may be 0.1% to 3% by mass, may be 0.12% to 1% by mass, or may be 0.15% to 0.5% by mass, based on the total amount of the cosmetic, from the viewpoint of achieving both the dispensability and the suppression of color separation at the application part at high levels.

In one or more embodiments, the aqueous liquid cosmetic may comprise as the component (B1), an anionic polymer (B1-L) having an average molecular weight (Mw) of 1,000 to 30,000 and an anionic polymer (B1-H) having an average molecular weight (Mw) of 50,000 to 1,000,000, from the viewpoint of achieving all the dispensability, the suppression of color separation at the application part, and the shading difference in the drawn lines. The mass ratio of these polymers, (B1/L)/(B1-H), may be 0.1/1 to 5/1, may be 1/5 to 3/1, or may be 1/3 to 5/3.

Examples of the nonionic dispersant include a polyoxyalkylene alkyl ether, a glycerin alkyl ether, a glycerin fatty acid ester, a polyglycerin fatty acid ester, a sorbitan fatty acid ester, and alkylene glycol adducts of those, a polyalkylene glycol fatty acid ester, a polyglycerin-modified silicone, and a polyether-modified silicone.

Examples of a water-soluble polymer other than the above-described dispersants include polyvinylpyrrolidone (PVP), polyvinyl alcohol (PVA), and a vinylpyrrolidone-vinyl acetate copolymer (VP-VA copolymer).

In one or more embodiments, the content of the component (B) in the aqueous liquid cosmetic may be 0.1% to 5% by mass, may be 0.2% to 4% by mass, or may be 0.3% to 3% by mass, based on the total amount of the cosmetic.

Component (C1)

The component (C1) is at least one inorganic salt selected from the group consisting of a chloride, a sulfate, a nitrate, a carbonate, and a phosphate. Examples of the component (C1) include chlorides such as sodium chloride, potassium chloride, magnesium chloride, calcium chloride, aluminum chloride, zinc chloride, and ammonium chloride; sulfates such as sodium sulfate, potassium sulfate, magnesium sulfate, aluminum sulfate, zinc sulfate, and ammonium sulfate; nitrates such as sodium nitrate, potassium nitrate, magnesium nitrate, calcium nitrate, aluminum nitrate, zinc nitrate, and ammonium nitrate; carbonates such as sodium carbonate, potassium carbonate, magnesium carbonate, and calcium carbonate; and phosphates such as sodium phosphate, potassium phosphate, and polyphosphates such as sodium metaphosphate and potassium metaphosphate. From the viewpoint of suppressing color separation at the application part, sodium chloride and magnesium chloride may be used.

From the viewpoint of achieving both the dispensability and the suppression of color separation at the application part at high levels, the content of the component (C1) in the aqueous liquid cosmetic may be 0.005% to 1% by mass, may be 0.01% to 0.8% by mass, may be 0.05% to 1% by mass, may be 0.06% to 0.8% by mass, may be 0.02% to 0.5% by mass, or may be 0.07% to 0.5% by mass, based on the total amount of the cosmetic.

When the aqueous liquid cosmetic contains a chloride as the component (C1), the content of the chloride may be 0.05% by mass or more or may be 0.1% by mass or more from the viewpoint of suppressing color separation at the application part. The content may be 1% by mass or less or may be 0.5% by mass or less from the viewpoint of dispensability, and the content may be 0.05% to 1% by mass or 0.1% to 0.5% by mass from the viewpoints of the suppression of color separation at the application part and the dispensability.

When the aqueous liquid cosmetic contains a polyphosphate as the component (C1), the content of the polyphosphate may be 0.005% by mass or more or may be 0.01% by mass or more from the viewpoint of suppressing color separation at the application part. The content may be 0.1% by mass or less or may be 0.05% by mass or less from the viewpoint of dispensability, and the content may be 0.005% to 0.1% by mass or 0.01% to 0.05% by mass from the viewpoints of the suppression of color separation at the application part and the dispensability.

Furthermore, from the viewpoint of suppressing color separation at the application part, the mass ratio of the component (C1) to the component (A1), [(C1)/(A1)], may be 1/2000 to 1/4, may be 1/200 to 1/4, may be 1/1000 to 1/5, may be 1/150 to 1/5, may be 1/900 to 1/6, may be 1/100 to 1/6, may be 1/800 to 1/7, or maybe 1/70 to 1/7.

When the aqueous liquid cosmetic contains a chloride as the component (C1), the mass ratio of the chloride to the component (A1), [(chloride)/(A1)], may be 1/1000 or more or may be 1/200 or more from the viewpoint of suppressing color separation at the application part. The content may be 1/4 or less or may be 1/10 or less from the viewpoint of dispensability, and the content may be 1/1000 to 1/4 or 1/200 to 1/10 from the viewpoints of the suppression of color separation at the application part and the dispensability.

When the aqueous liquid cosmetic contains a polyphosphate as the component (C1), the mass ratio of the polyphosphate to the component (A1), [(polyphosphate)/(A1)], may be 1/2000 or more or may be 1/1000 or more from the viewpoint of suppressing color separation at the application part. The content may be 1/50 or less or may be 1/150 or less from the viewpoint of dispensability, and the content may be 1/2000 to 1/50 or 1/1000 to 1/150 form the viewpoints of the suppression of color separation at the application part and the dispensability.

The aqueous liquid cosmetic may comprise the component (C1) and the above-mentioned component (B1-L) from the viewpoint of achieving both the dispensability and the suppression of color separation at the application part at high levels, and the mass ratio of these components, [(C1)/(B1-L)], may be 0.3/5 to 3/1, may be 0.4/5 to 2/1, or may be 0.1/1 to 1/1.

In the aqueous liquid cosmetic, other components that are usually used for cosmetic products in addition to each of the above-described components, for example, a powder other than the component (A), a surfactant, a film-forming agent, a humectant, a viscosity adjusting agent, an antiseptic agent, a pH adjusting agent, a chelating agent, an ultraviolet absorber, vitamins, aesthetic components, an oxidation inhibitor, a fragrance, and the like can be appropriately blended as necessary, to the extent that does not impair the effects of the present invention.

Examples of the powder other than the component (A) include white extender powders such as talc, muscovite, phlogopite, lepidolite, biotite, synthetic mica, sericite, synthetic sericite, kaolin, silicon carbide, bentonite, smectite, diatomaceous earth, aluminum silicate, aluminum magnesium metasilicate, calcium silicate, barium silicate, magnesium silicate, calcium carbonate, magnesium carbonate, hydroxyapatite, and boron nitride; synthetic resin powders such as a polyamide-based resin, a polyethylene-based resin, a polyacrylic resin, a polyester-based resin, a fluorine-based resin, a cellulose-based resin, a polystyrene-based resin, and a styrene-acrylic copolymerized resin; organic polymer resin powders such as a polypropylene-based resin and a urethane resin; organic low-molecular weight compound powders such as zinc stearate and N-acyl lysine; and natural organic powders such as a silk powder and a cellulose powder.

Regarding the surfactant, a hydrophilic nonionic surfactant, an anionic surfactant, a cationic surfactant, an amphoteric surfactant, and the like can be used.

Examples of the hydrophilic nonionic surfactant include a polyoxyalkylene alkyl ether, a glycerin alkyl ether, a glycerin fatty acid ester, a polyglycerin fatty acid ester, a sorbitan fatty acid ester, and alkylene glycol adducts of those; a polyalkylene glycol fatty acid ester, a polyglycerin-modified silicone, and a polyether-modified silicone. Examples of the anionic surfactant include an alkyl phosphate, a polyoxyalkylene alkyl ether phosphate, a sulfonate, and an alkyl sulfate. Examples of the cationic surfactant include an alkylamine salt and an alkyltrimethylammonium salt. Examples of the amphoteric surfactant include lecithin, a carbobetaine type amphoteric surfactant, a sulfobetaine type amphoteric surfactant, and an amino acid type amphoteric surfactant.

Examples of the film-forming agent include a film-forming polymer emulsion, PVP, PVA, and a VP/VA copolymer.

The polymer included in the film-forming polymer emulsion may be a water-insoluble polymer or copolymer including an alkyl (meth)acrylate monomer as a constituent unit. Examples of the constituent unit of the copolymer include a vinyl acetate monomer and a styrene monomer. In the case of a copolymer, the copolymer may be a random copolymer, a graft copolymer, a block copolymer, or a core-shell type copolymer.

Specific examples of the film-forming polymer emulsion include an alkyl acrylate copolymer emulsion, an alkyl acrylate-styrene copolymer emulsion, and an alkyl acrylate-vinyl acetate copolymer emulsion. The alkyl acrylate as used herein also includes an alkyl methacrylate. Regarding the film-forming polymer emulsion, a polymer emulsion containing water as a medium and having a solid content concentration of 30% to 60% by mass can be used.

Regarding the film-forming polymer emulsion, a commercially available product can be used. Examples of the alkyl acrylate copolymer emulsion include YODOSOL GH800F (sold under this name and manufactured by Akzo Nobel N.V., solid content concentration 45% by mass), YODOSOL GH810F (sold under this name and manufactured by Akzo Nobel N.V., solid content concentration 46% by mass), YODOSOL GH34F (sold under this name and manufactured by Akzo Nobel N.V., solid content concentration 42% by mass), and DAITOSOL 5000SJ (sold under this name and manufactured by Daito Kasei Kogyo Co., Ltd., solid content concentration 50% by mass). Examples of the alkyl acrylate-styrene copolymer emulsion include YODOSOL GH41F (sold under this name and manufactured by Akzo Nobel N.V., solid content concentration 45% by mass), DAITOSOL 5000STY (sold under this name and manufactured by Daito Kasei Kogyo Co., Ltd., solid content concentration 50% by mass), and EMUPOLY CE-119N (sold under this name by Nikko Chemicals Co., Ltd.). Examples of the alkyl acrylate-vinyl acetate copolymer emulsion include VINYSOL 2140L (sold under this name and manufactured by Daido Chemical Corporation, solid content concentration 45% by mass).

Regarding the film-forming polymer emulsion, one kind thereof can be used singly, or two or more kinds thereof can be used in combination.

In one or more embodiments, the content of the film-forming agent in the aqueous liquid cosmetic may be 2% to 20% by mass, may be 4% to 16% by mass, may be 6% to 12% by mass, or 7% to 10% by mass, in terms of the solid content concentration, based on the total amount of the cosmetic.

In one or more embodiments, the aqueous liquid cosmetic can be produced by dissolving or dispersing the above-mentioned component (A), component (B), component (C1), and water as well as other components as necessary, and uniformly stirring and mixing the components.

In one or more embodiments, the aqueous liquid cosmetic can be used as a makeup cosmetic such as an eyeliner, an eyebrow, an eyeshadow, or a mascara.

In one or more embodiments, the aqueous liquid cosmetic may be utilized in a cosmetic product. Example products include a pen-shaped product and a bottle-shaped product. The pen-shaped product may comprise a cosmetic accommodation part, such as a fiber bundle impregnated with a cosmetic or a filling part filled with a cosmetic, and an application part comprising a brush or a felt joined thereto. Examples of the pen-shaped product include a mechanical type from which the cosmetic is forcibly dispensed through application of force to the accommodation part by dialing or knocking and an automatic pen type from which a liquid cosmetic is dispensed by the action of its surface tension and a capillary phenomenon.

From the viewpoints of convenience in use and portability, the aqueous liquid cosmetic may be used in an automatic pen-shaped product according to one embodiment. The automatic pen-shaped product may employ a container, such as the liquid cosmetic container disclosed in Japanese Unexamined Patent Publication No. 2016-87094.

The automatic pen-shaped product may comprise an automatic pen-shaped applicator and the aqueous liquid cosmetic filled in the applicator.

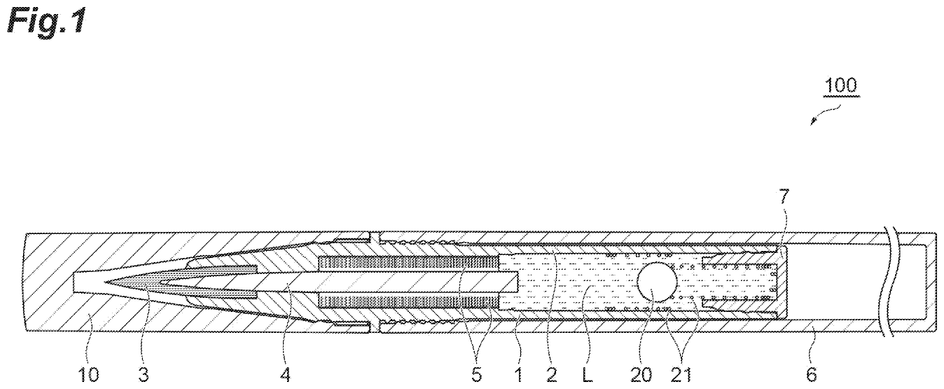

FIG. 1 is a schematic cross-sectional view showing an example pen-shaped cosmetic product 100. The overall shape of the pen-shaped cosmetic product 100 resembles a writing tool in an elongated narrow round-bar form. In some examples, the container comprises: a cylindrical container body 1; an accommodation part 2 disposed in the container body 1 so as to accommodate an aqueous liquid cosmetic L according to any embodiment described above; a brush 3 installed at a tip of the container body 1 for the application of the aqueous liquid cosmetic L in the accommodation part 2; a shaft-like relay wick 4 disposed in the container body 1 for connecting the inside of the accommodation part 2 with the brush 3; and a bellows component 5 in an approximately cylindrical shape installed around the relay wick 4. Additionally, a gripping cylinder 6 in a bottomed cylindrical shape is screwed to the container body 1 for detachable installation so that a user holding the container body 1 can easily apply the cosmetic. The shape of the container body 1 may be a cylindrical or rectangular tube, or some other shape.

In one or more embodiments of the pen-shaped cosmetic product 100, an automatic pen-shaped applicator may comprise an accommodation part 2, a brush 3 and a relay wick 4 wherein an aqueous liquid cosmetic according to one embodiment is filled in the accommodation part.

The container body 1 is, for example, formed of polypropylene (PP) and is configured to be a tapered cylindrical shape with a flange. A rear end face of the flange part disposed on an outer peripheral surface of the container body 1 abuts on a front-end face of the gripping cylinder 6 screwed into the container body 1. A front-end face of the flange part abuts on an open end face of a cap 10 installed on the container body 1. An opening at a rear end of the container body 1 is closed with a bottomed cylindrical tale plug 7 inserted therein.

The bellows component 5 is intended for controlling the flow rate of the aqueous liquid cosmetic L and has grooves (bellows) containing the aqueous liquid cosmetic L. A cylindrical rear-end part of the bellows component 5 fits in a recess of an inner peripheral face of the container body 1 so that the bellows component 5 is installed within the container body 1. The accommodation part 2 is formed between the rear-end part of the bellows component 5 and the tail plug 7, and the aqueous liquid cosmetic L is filled in the accommodation part 2.

The relay wick 4 is, for example, formed of an acrylic resin and extends in the axial direction to pass through a tubular hole of the bellows component 5. A front-end side of the relay wick 4 fits into a front-end side of the bellows component 5 so that the relay wick 4 is installed within the bellows component 5. The relay wick 4 connects the inside of the accommodation part 2 to the brush 3, while its rear-end side part enters into the inside of the accommodation part 2 and its front-end side part enters into the brush 3. The relay wick 4 allows the aqueous liquid cosmetic L to be sucked from the accommodation part 2 by a capillary phenomenon and to be supplied to the brush 3.

A brush serves as an application tool in the pen-shaped cosmetic product 100 shown in FIG. 1, but it may be replaced by a felt tip or a urethane tip.

A bottomed cylindrical cap 10 is detachably installed at the front-end side of the container body 1 by fitting to protect the brush 3.

The accommodation part 2 of the pen-shaped cosmetic product 100 accommodates a stirring element 20 that is movable in the axial direction and a coil spring 21 that is extendable in the axial direction, together with the aqueous liquid cosmetic L. In FIG. 1, the stirring element 20 is depicted as a sphere; however, the element may be a polyhedron, a cone or other shape.

The coil spring 21 is an integrally formed spring including a plurality of spring parts having different diameters (e.g., two spring parts having different diameters) integrally connected in the axial direction, which may be formed of SUS (Steel Use Stainless), for example. The coil spring 21 comprises a small-diameter spring part having a diameter smaller than that of the stirring element 20 in a rear half part thereof and a large-diameter spring part having a diameter larger than that of the stirring element 20 continuously, the latter of which spring parts is adjacent to the forward axial direction of the small-diameter spring.

When the pen-shaped cosmetic product 100 is shaken by a user, the stirring element 20 that is movable in the axial direction in the large-diameter spring of the coil spring 21 moves in the axial direction. Thus, the aqueous liquid cosmetic L is stirred by the movement of the stirring element 20.

In the pen-shaped cosmetic product 100 thus configured, the aqueous liquid cosmetic L in the accommodation part 2 flows toward the brush 3 at a front-end side of the container through the rely wick 4, whereby it is provided to a user for application with the brush 3. The pen-shaped cosmetic product 100 includes the stirring element 10 and the coil spring 21, thereby allowing the aqueous liquid cosmetic L to efficiently flow toward the brush 3. This configuration may be changed such that it neither includes the stirring element 10 nor the coil spring 21.

An example pen-shaped cosmetic product according to one embodiment has been described above by referring to the cosmetic product having a so-called direct liquid type structure. However, other types of structures may be used. For example, a container having a so-called wadding structure may be used, which omits the bellows components 5, the stirring element 20 and the coil spring 21 in the pen-shaped cosmetic product 100 and which has an accommodation part 2 accommodating a wadding component impregnated with the aqueous liquid cosmetic L and sucks the aqueous liquid cosmetic L therefrom to supply to the brush 3 through a rear end part of the relay wick 4 entering into an inner part of the wadding component.

### ADDITIONAL EXAMPLE EMBODIMENTS

Hereinafter, the present invention will be described more specifically by way of Examples; however, the present invention is not intended to be limited to the following Examples.

<Production of aqueous liquid cosmetic>

(Examples 1 to 15 and Comparative Examples 1 to 3)

The various components shown in Tables 1 to 4 were mixed with a Disper at the proportions (% by mass) indicated in the same tables, and each aqueous liquid cosmetic was obtained. In the tables, the values for the polymer emulsion, the anionic dispersant, and the nonionic dispersant represent the blending amounts of solid contents.

Regarding each of the components shown in Tables 1 to 4, materials shown below were used.

Na polyaspartate: Na polyaspartate (manufactured by Ajinomoto Co., Inc. and sold under the name of “AQUADEW SPA-30B”, average molecular weight (Mw) 4,000)

Na polyacrylate: Na polyacrylate (manufactured by Toagosei Co., Ltd. and sold under the name of “ARON T-50”, average molecular weight (Mw) 6,000)

Na polycarboxylate: Na polyacrylate (manufactured by Toagosei Co., Ltd. and sold under the name of “ARON A-6330”, average molecular weight (Mw) 10,000)

Alkyl acrylate copolymer: Alkyl acrylate copolymer (manufactured by BASF SE and sold under the name of “Luvimer 100-P”, average molecular weight (Mw) 70,000)

Nonionic dispersant-1: Beheneth-30 (polyoxyethylene behenyl ether, manufactured by Nikko Chemicals Co., Ltd. and sold under the name of “NIKKOL BB-30”)

Film-forming agent-1: (Acrylate/ethylhexyl acrylate) copolymer (manufactured by Daito Kasei Kogyo Co., Ltd. and sold under the name of “DAITOSOL 5000SJ”)

Film-forming agent-2: PVP K-90 (polyvinylpyrrolidone, manufactured by Ashland Specialty Ingredients G.P. and sold under the name of “PVP K-90”)

<Viscosity of aqueous liquid cosmetic>

For a sample at 25° C., viscosity was measured using a Brookfield type viscometer (BM type) and a BL adaptor under the conditions of a speed of rotation of 12 rpm. The measuring time was set to 1 minute.

<Evaluation of aqueous liquid cosmetic>

For the aqueous liquid cosmetics obtained as described above, dispensability, color separation at the application part, and shading difference in the drawn lines were evaluated by the following evaluation methods.

(Dispensability)

An aqueous liquid cosmetic obtained as described above was filled in an accommodation part of an automatic pen shaped liquid eyeliner container (application tool: nylon tip, relay wick: acrylic resin) having a structure similar to that of the container shown in FIG. 1 to produce a filled product.

Container-filled products were used to write on the back of the hand, the satisfactoriness of dispensability at the time of applying three lines each measuring 2 mm in width and 4 cm in length was visually inspected, and the dispensability were evaluated according to the following evaluation criteria.

[Evaluation criteria]

A: A sufficient liquid amount is dispensed, and the drawn lines are uniform.

B: An acceptable liquid amount is dispensed, and the drawn lines are almost uniform.

C: Because the liquid amount being dispensed is small, the drawn lines are slightly uneven.

D: The liquid amount being dispensed is small, and the drawn lines are scratchy.

(Color separation at application part and shading difference (hue difference) in drawn lines)

Container-filled products were prepared as described above and were stored at room temperature for two weeks in a state of being placed sideways and fixed. The application tool (application part) after storage was checked by visual inspection, and color separation was evaluated by the following evaluation criteria.

[Evaluation criteria: color separation at application part]

A: There is no change from before storage, the application part is uniform, and no color separation is not observed.

B: There is a slight change from before storage, but the application part is almost uniform, and color separation is hardly observed.

C: There is a change from before storage, and slight color separation is observed at the application part.

D: There is an obvious change from before storage, and complete color separation is observed at the application part.

Container-filled products were prepared as described above, and some of them were placed vertically with the pen tip (application tool) facing upward, while the others were placed vertically with the pen tip facing downward, after which the products were respectively stored at room temperature for two weeks. The filled products that had been stored for two weeks were used to write on the back of the hand, and the shading difference (hue difference) in the drawn lines were evaluated according to the following evaluation criteria.

[Evaluation criteria: shading difference (hue difference) in drawn lines]

A: No hue difference is observed at all between the products stored facing upward and the products stored facing downward.

B: Hue difference is hardly observed between the products stored facing upward and the products stored facing downward.

C: A slight hue difference is observed between the products stored facing upward and the products stored facing downward.

D: A considerable hue difference is observed between the products stored facing upward and the products stored facing downward.

TABLE 1

| | | | Example 1 | Example 2 | Example 3 | Example 4 | Example 5 |
|---|---|---|---|---|---|---|---|
| (A) | (A1) | Prussian blue | 9 | — | 9 | — | 9 |
| Pigment | | Carmine | — | 4.5 | — | 4.5 | — |
| | Carbon black | | 0.5 | 0.5 | 0.5 | 0.5 | 0.5 |
| (B) | (B1) Anionic | Na polyaspartate | 0.5 | 0.5 | 0.5 | 0.5 | 0.5 |
| Dispersant | dispersant | Alkyl acrylate copolymer | 1 | 1.5 | 1 | 1.5 | 1 |
| Inorganic | (C1) | Sodium chloride | 0.3 | 0.3 | 0.05 | 1 | — |
| salt | | Magnesium chloride | — | — | — | — | 0.3 |

| | | | | | |
|---|---|---|---|---|---|
| Film-forming agent-1 | 8 | 8 | 8 | 8 | 8 |
| Film-forming agent-2 | 0.5 | 0.5 | 0.5 | 0.5 | 0.5 |
| pH adjusting agent (potassium hydroxide) | 0.04 | 0.04 | 0.04 | 0.04 | 0.04 |
| Purified water | Balance | Balance | Balance | Balance | Balance |
| Anhydrous ethanol | 3 | 3 | 3 | 3 | 3 |
| 1,3-Butylene glycol | 6 | 6 | 6 | 6 | 6 |
| Antiseptic agent | 0.8 | 0.8 | 0.8 | 0.8 | 0.8 |
| (phenoxyethanol, Na dehydroacetate) | | | | | |
| Total (mass %) | 100 | 100 | 100 | 100 | 100 |
| Component (C1) (mass %) | 0.3 | 0.3 | 0.05 | 1 | 0.3 |
| Mass ratio [(C1)/(A1)] | 0.03 | 0.07 | 0.006 | 0.22 | 0.03 |
| Viscosity (mPa · s) | 16 | 15 | 12 | 18 | 15 |
| Dispensability | A | A | A | B | A |
| Color separation at application part | A | A | B | A | A |
| Shading difference (hue difference) in drawn lines | A | A | B | A | A |

TABLE 2

| | | | Example 6 | Example 7 | Example 8 | Example 9 | Example 10 |
|---|---|---|---|---|---|---|---|
| (A) | (A1) | Prussian blue | 9 | 9 | 9 | 9 | 3 |
| Pigment | Carbon black | | 0.5 | 0.5 | 0.5 | 0.5 | — |
| | Titanium oxide | | — | — | — | — | 6 |
| (B) | (B1) | Na polyaspartate | — | — | 0.1 | 5 | 0.5 |
| Dispersant | Anionic | Na polyacrylate | 0.5 | — | — | — | — |
| | dispersant | Na polycarboxylate | — | 0.5 | — | — | — |
| | | Alkyl acrylate copolymer | 1 | 1 | 1 | 1 | 0.3 |
| | Nonionic dispersant-1 | | — | — | — | — | 0.2 |
| Inorganic salt | (C1) | Sodium chloride | 0.3 | 0.3 | 0.3 | 0.3 | 0.3 |

| | | | | | |
|---|---|---|---|---|---|
| Film-forming agent-1 | 8 | 8 | 8 | 8 | 8 |
| Film-forming agent-2 | 0.5 | 0.5 | 0.5 | 0.5 | 0.5 |
| pH adjusting agent (potassium hydroxide) | 0.04 | 0.04 | 0.04 | 0.04 | 0.04 |
| Purified water | Balance | Balance | Balance | Balance | Balance |
| Anhydrous ethanol | 3 | 3 | 3 | 3 | 3 |
| 1,3-Butylene glycol | 6 | 6 | 6 | 6 | 6 |
| Antiseptic agent | 0.8 | 0.8 | 0.8 | 0.8 | 0.8 |
| (phenoxyethanol, Na dehydroacetate) | | | | | |
| Total (mass %) | 100 | 100 | 100 | 100 | 100 |
| Component (C1) (mass %) | 0.3 | 0.3 | 0.3 | 0.3 | 0.3 |
| Mass ratio [(C1)/(A1)] | 0.03 | 0.03 | 0.03 | 0.03 | 0.1 |
| Viscosity (mPa · s) | 15 | 16 | 12 | 18 | 18 |
| Dispensability | A | A | A | B | A |
| Color separation at application part | A | A | B | A | A |
| Shading difference (hue difference) in drawn lines | A | A | B | A | A |

TABLE 3

| | | | Example 11 | Example 12 | Example 13 | Example 14 | Example 15 |
|---|---|---|---|---|---|---|---|
| (A) | (A1) | Prussian blue | 9 | 9 | 9 | 9 | 9 |
| Pigment | Carbon black | | 0.5 | 0.5 | 0.5 | 0.5 | 0.5 |
| (B) | (B1) Anionic | Na polyaspartate | 0.5 | 0.5 | 0.5 | 0.5 | 0.5 |
| Dispersant | dispersant | Alkyl acrylate copolymer | 1 | — | 1 | 1 | 1 |
| | Nonionic dispersant-1 | | — | 2 | 1 | 1 | 1 |
| Inorganic | (C1) | Sodium chloride | 0.1 | 0.3 | — | — | — |
| salt | | Sodium metaphosphate | — | — | 0.01 | 0.05 | 0.1 |

| | | | | | |
|---|---|---|---|---|---|
| Film-forming agent-1 | 8 | 8 | 8 | 8 | 8 |
| Film-forming agent-2 | 0.5 | 0.5 | 0.5 | 0.5 | 0.5 |
| pH adjusting agent (potassium hydroxide) | 0.04 | 0.04 | 0.04 | 0.04 | 0.04 |
| Purified water | Balance | Balance | Balance | Balance | Balance |
| Anhydrous ethanol | 3 | 3 | 3 | 3 | 3 |
| 1,3-Butylene glycol | 6 | 6 | 6 | 6 | 6 |
| Antiseptic agent | 0.8 | 0.8 | 0.8 | 0.8 | 0.8 |
| (phenoxyethanol, Na dehydroacetate) | | | | | |
| Total (mass %) | 100 | 100 | 100 | 100 | 100 |
| Component (C1) (mass %) | 0.1 | 0.3 | 0.01 | 0.05 | 0.1 |
| Mass ratio [(C1)/(A1)] | 0.01 | 0.03 | 0.001 | 0.006 | 0.011 |
| Viscosity (mPa · s) | 14 | 20 | 12 | 13 | 13 |
| Dispensability | A | B | A | A | A |
| Color separation at application part | A | A | B | B | C |
| Shading difference (hue difference) in drawn lines | A | A | B | B | C |

TABLE 4

| | | | Comparative Example 1 | Comparative Example 2 | Comparative Example 3 |
|---|---|---|---|---|---|
| (A) | (A1) | Prussian blue | 9 | 9 | 9 |
| Pigment | | | | | |
| | Carbon black | | 0.5 | 0.5 | 0.5 |
| (B) | (B1) | Na polyaspartate | 0.5 | — | 0.5 |
| Dispersant | Anionic | Alkyl acrylate copolymer | 1 | 1 | 1 |
| | dispersant | | | | |

| | | | |
|---|---|---|---|
| Film-forming agent-1 | 8 | 8 | 8 |
| Film-forming agent-2 | 0.5 | 0.5 | 0.5 |
| Na citrate | — | — | 0.3 |
| pH adjusting agent (potassium hydroxide) | 0.04 | 0.04 | 0.04 |
| Purified water | Balance | Balance | Balance |
| Anhydrous ethanol | 3 | 3 | 3 |
| 1,3-Butylene glycol | 6 | 6 | 6 |
| Antiseptic agent (phenoxyethanol, Na dehydroacetate) | 0.8 | 0.8 | 0.8 |
| Total (mass %) | 100 | 100 | 100 |
| Component (C1) (mass %) | — | — | — |
| Mass ratio [(C1)/(A1)] | 0 | 0 | 0 |
| Viscosity (mPa·s) | 10 | 8 | 19 |
| Dispensability | A | C | B |
| Color separation at application part | D | D | C |
| Shading difference (hue difference) in drawn lines | D | D | D |

### REFERENCE SIGNS LIST

1: container main body, 2: accommodation part, 3: brush (application tool), 20: stirring bar, 21: coil spring (small-diameter spring part and large-diameter spring part), 100: pen-shaped cosmetic product, L: aqueous liquid cosmetic.

What is claimed is:

1. An aqueous liquid cosmetic comprising:
a pigment including at least one selected from the group consisting of Prussian blue and lakes of natural colorants;
a dispersant; and
an inorganic salt including at least one selected from the group consisting of a chloride, a sulfate, a nitrate, a carbonate, and a phosphate,
wherein the cosmetic has a viscosity of 1 to 50 mPa·s at 25° C.

2. The aqueous liquid cosmetic according to claim 1,
wherein the inorganic salt comprises a chloride, and
a content of the chloride is 0.05% to 1% by mass based on a total amount of the cosmetic.

3. The aqueous liquid cosmetic according to claim 1,
wherein the inorganic salt comprises a polyphosphate, and
a content of the polyphosphate is 0.005% to 0.1% by mass based on a total amount of the cosmetic.

4. The aqueous liquid cosmetic according to claim 1, wherein the dispersant comprises an anionic dispersant.

5. The aqueous liquid cosmetic according to claim 4,
wherein the anionic dispersant is an anionic polymer having an average molecular weight (Mw) of 1,000 to 30,000, and
a mass ratio of the inorganic salt to the anionic polymer is 0.3/5 to 3/1.

6. The aqueous liquid cosmetic according to claim 4, wherein the anionic dispersant comprises:
an anionic polymer having an average molecular weight (Mw) of 1,000 to 30,000; and
an anionic polymer having an average molecular weight (Mw) of 50,000 to 1,000,000.

7. The aqueous liquid cosmetic according to claim 1, wherein the pigment further comprises at least one metal oxide selected from the group consisting of titanium oxide, red iron oxide, yellow iron oxide, black iron oxide, and titanium black.

8. A pen-shaped cosmetic product comprising an automatic pen-shaped applicator and the aqueous liquid cosmetic according to claim 1 filled in the applicator.
